(19) United States
(12) Patent Application Publication
(10) Pub. No.: US 2002/0082590 A1
(43) Pub. Date: Jun. 27, 2002

(54) Topography-guided opthalmic ablation and eye-tracking

(76) Inventor: Frederick J. Potgieter, Pretoria (ZA)

Correspondence Address: TRASK BRITT, P.O. BOX 2550, SALT LAKE CITY, UT, 84110, US

(21) Appl. No.: 09/993,753

(22) Filed: Nov. 14, 2001

Related U.S. Application Data
(60) Provisional application No. 60/248,452, filed on Nov. 14, 2000.

(51) Int. Cl. A61B 18/18; A61B 19/00

(52) U.S. Cl. 606/4; 606/5; 606/11; 128/898

(57) ABSTRACT

Systems and methods for topography-guided ophthalmic ablation and eye-tracking. A topographic map of the surface of an eye is generated. A reference pattern on the surface of the eye, such as a staining substance applied to points on the eye, is correlated with the topographic map. The eye surface pattern is continuously tracked and the correlation adjusted. Ablation of the cornea may be performed based on the correlation as it is adjusted in real time.

### RELATED APPLICATIONS

This application claims the benefit under 35 U.S.C. §119(e) of U.S. Provisional Application No. 60/248,452, filed Nov. 14, 2001, which is incorporated herein by reference in its entirety.

### TECHNICAL FIELD

The invention relates to topography-guided ophthalmic ablation and eye-tracking. More generally, however, some embodiments of the invention have the following aspects: a method of notionally superimposing a topographic map of an eye upon a cornea of an eye; an automated ophthalmic surgery system for conducting topography-guided ablation of an eye; a data compilation system for compiling data useful for topography-guided ablation of an eye; a method of performing an automated ablation treatment of an eye in register with the eye concerned; a method of tracking an eye; an eye-tracking system; a database; a digital data storage medium; and an ophthalmic applicator.

### BACKGROUND

Eye-tracking has a number of applications. In the field of ophthalmology, for example, eye-tracking is an integral aspect of laser refractive procedures such as photorefractive keratectomy (PRK) and laser in situ keratomileusis (LASIK). In these procedures, eye-tracking systems are used to guide excimer lasers in the application of calculated treatments to corneas. However, using conventional techniques, custom corneal ablation has not yet been satisfactorily achievable. Custom ablations are required, for example, to correct irregular astigmatism. Patients with irregular astigmatism who are treated only for sphere keep their residual astigmatism or achieve only 20/40 vision. Trial protocols may define this as successful, but it still falls short of patient expectations.

Some progress towards custom ablations has been made by cooperation between topographer manufacturing companies and excimer laser manufacturing companies. Thus, for example, Orbtek topographers have been linked with LaserSight excimer lasers. Technomed's C-Scan topography unit has been combined with Bausch & Lomb Surgical's keracor 217 excimer laser to form the Technolas Topolink configuration. These systems have eye-tracking and an effectively working scanning laser, but have not yet achieved an accurate link between a topographic map for a particular surgery, and eye-tracking during that surgery.

The problem can be stated differently as a lack of real time targeting to follow the eye as it moves. In a report dated Dec. 1, 1998 appearing on the Internet at URL: “http//www.usaeyes.org/articles/topography.htm”, Michael C Knorz, MD is quoted as follows: “The topographic map has to remain superimposed on the cornea as the eye moves, and researchers are not yet close to that goal.”

Some of the difficulties which researchers have experienced relate to conventional methods of eye-tracking. For example, methods of eye-tracking for guidance of lasers performing ablations have to date included tracking a mathematically determined center of a pupil, tracking an edge of the pupil (the LADARVision system), or tracking the inner wall of a suction ring applied to the periphery of the cornea.

There are shortcomings associated with these and other conventional methods of eye-tracking, particularly when required for linking the topography of a laser irradiation zone with a laser by way of the eye-tracker. Thus, in the context of refractive eye surgery, conventional systems of eye-tracking do not permit accurate linking of a topographic map of an eye with a laser. This shortcoming has the consequence that only approximately 40% of custom treatments performed using conventional current systems are successful. In the majority of cases, results include limited improvement of vision, no improvement, or an exacerbation of vision resulting from lack of registration of the topographic map with the eye. Exacerbation of vision can occur because, for example, a treatment calculated to ablate an elevated region of a cornea can actually create a recess in the cornea if performed out of register with the elevated region, thereby increasing the irregularity of the eye.

In summary, processed information which is obtained from a topographer must be matched point for point precisely with corresponding points on the cornea during treatment of an eye with a laser. Until now, there has been no method of accurately coordinating information from the topographer with the treatment effected by the laser.

### DISCLOSURE OF INVENTION

Accordingly, there is provided, in a first aspect of the invention, a method of notionally superimposing a topographic map of an eye upon a cornea of an eye, which method includes the steps of: applying a predetermined reference pattern to the eye; scanning the eye thereby to derive a topographic map of the eye and capture data representing the location and/or orientation of the pattern on the eye; correlating the location and/or orientation of the pattern with corresponding coordinates of the topographic map representing the pattern's location and/or orientation on the topographic map, and/or with data (e.g., elevation data) associated with the coordinates on the topographic map; tracking the pattern continually, thereby to produce a real-time, intermittent update of the location and/or orientation of the pattern; and notionally projecting the location and/or orientation of the topographic map upon the eye by mapping the coordinates of the topographic map that represent the pattern's location and/or orientation on the topographic map, to the physical location and/or orientation of the pattern.

For purposes of this specification, the term “superimposing” means correlating coordinates of a topographic map of an eye, and data (e.g., elevation data, placido data, and wavefront data) for each set of coordinates of the map, with physical loci of the eye represented by the coordinates.

The predetermined reference pattern is preferably an artificial pattern applied to the eye. The pattern may be in the form of a plurality of discrete reference markings, and the method may thus include tracking the plurality of markings, or specific portions of the markings. In one embodiment of the invention, three markings are applied in a non-colinear arrangement. In another embodiment, only two markings are used. The pattern may include at least one marking recognizable by optical character recognition (OCR) hardware, digital signal processing (DSP) hardware, and/or software means. For example, the marking or markings may be members of a recognizable character set.

The markings may be applied by applying a staining substance to the eye, for example, to the cornea of the eye. Masking means such as a stencil, and/or impression means such as a stamp-like applicator, may be used to imprint the staining substance on the eye. Instead, or in addition, the markings may be formed from semi-permanent materials. For example, the markings may be in the form of temporary sutures, or one or more printed patches or templates.

For the purpose of this specification, the term “staining substance” is to be interpreted widely to include any material distinguishable by imaging systems. The term is not restricted to stains and dyes emitting light in the visible spectrum, but also includes those emitting or capable of emitting radiation in the infrared and ultra-violet regions of the spectrum, as well as radio-isotopic tracers, etc. The staining substance may be methylene blue, or any other suitable physiologically acceptable staining substance.

Scanning the eye to capture data representing the location and/or orientation of the pattern on the eye may include capturing an image of the eye, for instance, a bitmap or other digital image, photograph or video still. Image processing means may then be employed to identify the pattern in the image of the eye, for example, with the aid of optical character recognition (OCR) hardware, DSP hardware, and/or software means.

Tracking the predetermined reference pattern continually may be performed by capturing successive images of the eye and identifying in the images the predetermined reference pattern and its location and/or orientation. Such identification may be performed with the aid of optical character recognition (OCR) hardware, DSP hardware, and/or software means, where the pattern is or includes a character recognizable by, for example, OCR means. The images may be captured at a frequency of 10 images per second, or higher.

Notionally projecting the location and/or orientation of the topographic map upon the eye is typically conducted continually and automatically with the aid of data processing means such as computer hardware and software.

Methods as described above may be employed in topography-guided automated ablation (e.g., laser ablation) of an eye, in order to provide a reference database (in the form of the superimposed topography) from which accurate data relating to an appropriate ablation treatment for a particular locus of an eye may be accessed.

Therefore, according to a further aspect of the invention, there is provided an automated ophthalmic surgery system for conducting topography-guided ablation of an eye, which includes scanning means configured to derive a topographic map of an eye, and capture an image of the eye; image processing means configured to (i) identify a predetermined reference pattern in the image of the eye, (ii) correlate the location and/or orientation of the pattern with corresponding coordinates of the topographic map representing the pattern's position and/or orientation on the topographic map, and/or with data (e.g. elevation data) associated with the coordinates on the topographic map, and (iii) compile resulting correlation data; eye-tracking means configured to track the predetermined reference pattern, for example by deriving successive images of the eye and identifying in the images the predetermined reference pattern and its location and/or orientation; and guidance means adapted to guide ophthalmic ablation means in the application of surgical treatment to a locus of an eye by (i) determining the topographic map coordinates of the locus relative to those of the predetermined reference pattern by means of the correlation data produced by the image processing means, (ii) accessing precalculated treatment data for the topographic map coordinates from a treatment data file, and (iii) guiding the ablation means to perform the precalculated treatment at the locus. The ophthalmic ablation means is typically, though not necessarily exclusively, a laser system such as an excimer laser system.

Again, the predetermined reference pattern is preferably an artificial pattern, as hereinbefore described, which has been applied to the eye, for example, with the aid of a staining substance, temporary sutures, printed patches and/or templates. Instead or in addition, the predetermined reference pattern may be or may include at least one natural pattern (or a portion of such a natural pattern) defined by a feature of the eye. As examples, the natural pattern may be a pattern of conjunctival vessels or of iris nevi. The image processing means and the eye-tracking means may be configured to identify such natural patterns or portions thereof. Existing pattern recognition systems such as those conventionally employed for identification of persons based on unique and distinctive physiological patterns (such as their fingerprints or iris nevi patterns) could be harnessed, with appropriate developments, to provide the identification of the natural patterns. The advantage of using such systems for identification of patterns in accordance with the invention, is that these systems employ a limited number of criteria and points of correlation in order to establish a match between patterns. As a result, such systems could be harnessed to identify a given pattern rapidly and efficiently. By employing such systems in an eye-tracking means, rapid updates of the location of the reference pattern will be facilitated with limited processing requirements, leading in turn to better real-time tracking of the reference pattern and guidance of the ophthalmic ablation means (e.g., the laser system). Eye-tracking for purposes of the invention may then involve analysis of, for example, an iris in order to localize specific characteristics of the iris, followed by tracking of those specific characteristics.

The reference pattern, whether artificial or natural, may be in the form of a plurality of discrete reference markings. Thus, the image processing means and the eye-tracking means may be configured to identify the plurality of discrete reference markings, or specific portions of the markings. The reference pattern may include at least one reference marking recognizable by optical character recognition (OCR) hardware, DSP hardware, and/or software means. For example, the marking or markings may be characters of a recognizable character set. The surgery system may thus include OCR means for recognizing characters forming part of the pattern, or portions of such characters.

The scanning means and the eye-tracking means may be configured to capture images in the form of bitmap or other digital images, photographs or video stills of the eye. The eye-racking means preferably captures images at a frequency of 10 images per second, or higher. The scanning means is typically a development of a conventional topographer, equipped to capture an image of an eye together with a topographic map thereof.

The invention further extends to a data compilation system for compiling data useful for topography-guided ablation of an eye, which includes scanning means configured to derive a topographic map of an eye and capture an image of the eye; and image processing means configured to (i) identify a predetermined reference pattern in the image of the eye, (ii) correlate the location and/or orientation of the pattern with corresponding coordinates of the topographic map representing the pattern's position and/or orientation on the topographic map, and/or with data (e.g., elevation data) associated with the coordinates on the topographic map, and (iii) compile resulting correlation data.

As previously described, the predetermined reference pattern is preferably an artificial pattern which has been applied to the eye, for instance, with the aid of a staining substance, temporary sutures, printed patches and/or templates. Instead or in addition, the predetermined reference pattern may be or may include at least one natural pattern (or a portion of such a natural pattern) defined by a feature of the eye, as hereinbefore described. The image processing means may be configured to identify such natural patterns or portions thereof. As previously discussed, existing pattern recognition systems such as those conventionally employed for identification of persons based on unique and distinctive physiological patterns (such as their fingerprints or iris nevi patterns) could be harnessed, with appropriate developments, to provide the identification of the natural patterns.

The reference pattern, whether artificial or natural, may be in the form of a plurality of discrete reference markings. Thus, the image processing means may be configured to identify the plurality of discrete reference markings, or specific portions of the markings. The pattern may include at least one reference marking recognizable by OCR hardware, DSP hardware, and/or software means. For example, the marking or markings may be characters of a recognizable character set. The data compilation system may thus include OCR or DSP means for recognizing characters forming part of the pattern, or portions of such characters.

The scanning means may be configured to capture images in the form of bitmap or other digital images, photographs or video stills of the eye. The scanning means is typically a development of a conventional topographer, equipped to capture an image of an eye together with a topographic map thereof.

In accordance with another aspect of the invention there is provided a method of performing an automated ablation treatment of an eye in register with the eye concerned, which includes the steps of calculating a treatment applicable at a locus of an eye corresponding to coordinates on a topographic map, and compiling a laser-shot file correlating data relating to the treatment with the coordinates; correlating the location and/or orientation of a predetermined reference pattern on the surface of an eye with corresponding coordinates of the topographic map representing the pattern's position and/or orientation on the topographic map, and/or with data (e.g., elevation data) associated with the coordinates on the topographic map; identifying the predetermined reference pattern on the surface of the eye and continually tracking its movement thereby to track notional movement of the topographic map and, consequently, the locus of the eye; and performing the treatment calculated for the locus, at the locus.

This method preferably includes applying the pre-determined reference pattern to the eye prior to the ablation treatment. Instead or in addition, the predetermined reference pattern may be or may include at least one natural pattern (or a portion of such a natural pattern) defined by a feature of the eye, as discussed above. The reference pattern, whether artificial or natural, may be in the form of a plurality of discrete reference markings. The pattern may include at least one marking recognizable by OCR hardware, DSP hardware, and/or software means. For example, the marking or markings may be members of a recognizable character set.

The markings may be applied by applying a staining substance to the cornea of the eye. Masking means such as a stencil, and/or impression means such as a stamp-like applicator, may be used to imprint the staining substance on the eye. As previously described, the markings may instead or additionally be formed from semi-permanent materials.

The step of correlating the location and/or orientation of the pattern on the surface of an eye with corresponding coordinates of the topographic map may include: capturing an image of the eye, for example, a bitmap or other digital image, photograph, or video still; and employing image processing means to (i) identify the pattern in the image of the eye, (ii) correlate the location and/or orientation of the pattern with corresponding coordinates of the topographic map representing the pattern's position and/or orientation on the topographic map, and/or with data (e.g., elevation data) associated with the coordinates on the topographic map, and (iii) compile resulting correlation data.

Identification of the pattern in the image of the eye may be performed with the aid of OCR hardware, DSP hardware, and/or software means, particularly where the pattern is artificial. Where the pattern is natural, existing pattern recognition systems such as those conventionally employed for identification of persons based on unique and distinctive physiological patterns (such as their fingerprints or iris nevi patterns) could be harnessed, with appropriate developments, to provide identification of the pattern.

The step of identifying the predetermined reference pattern on the surface of the eye and continually tracking its movement may be performed by capturing successive images of the eye and identifying in the images the predetermined reference pattern and its location and/or orientation. Where the pattern is in the form of a plurality of markings, tracking movement of the pattern may include identifying and tracking the plurality of markings, or specific portions of the markings. Again, such identification may be performed with conventional OCR means, DSP means, and/or pattern recognition systems, as hereinbefore described. The images may be captured at a frequency of 10 images per second, or higher.

The treatment calculated for a particular locus is typically performed by a laser ablation system, such as an excimer laser ablation system.

According to a further aspect of the invention there is provided a method of tracking an eye which includes the steps of configuring an eye-tracking system to recognize at least one pre-determined reference pattern; applying the pattern directly to a surface of an eye; and continually tracking the pattern with the eye-tracking system.

According to a further aspect of the invention there is provided an eye-tracking system which is pre-configured to recognize at least one predetermined reference pattern on the surface of an eye, and gather information regarding the location and/or orientation of the pattern.

For both the method of tracking an eye and the eye-tracking system, the predetermined reference pattern is preferably an artificial pattern applied to the eye. The pattern may be in the form of a plurality of discrete reference markings, and the method of tracking the eye may thus include tracking the plurality of markings, or specific portions of the markings. The pattern may include at least one marking recognizable by OCR hardware, DSP hardware, and/or software means. For example, the marking or markings may be members of a recognizable character set.

In the method of tracking an eye, applying the pattern directly to the surface of the eye may include applying a staining substance to the eye, e.g. to the cornea of the eye. Masking means such as a stencil, and/or impression means such as a stamp-like applicator, may be used to imprint the staining substance on the eye. As previously described, the markings may instead or additionally be formed from semi-permanent materials.

Configuring the eye-tracking system to recognize the pattern may include configuring the system with OCR hardware and/or software means.

Continually tracking the pattern with the eye-tracking system may be performed by capturing successive images of the eye and identifying in the images the pattern and its location and/or orientation. Such identification may be performed with the aid of conventional OCR means and/or pattern recognition systems, as hereinbefore described. The images may preferably be captured at a frequency of 10 images per second, or higher.

As regards the eye-tracking system, the system may be pre-configured with OCR hardware and/or software means and/or a pattern recognition system as described above. The eye-tracking system may, further, be pre-configured with means for capturing successive images of an eye, for interpretation by the OCR means and/or pattern recognition system.

According to a further aspect of the invention there is provided a database comprising data representative of a location and/or orientation of a predetermined reference pattern on a surface of an eye, defined with reference to coordinates of a topographic map of the eye. The predetermined reference pattern is preferably an artificial pattern as hereinbefore described. It may, however, be a natural pattern as hereinbefore described.

According to a further aspect of the invention there is provided a digital data storage medium (e.g., a magnetic or optical storage medium such as a magnetic disk or compact disk) on which is stored data representative of a location and/or orientation of a predetermined reference pattern on a surface of an eye, defined with reference to coordinates of a topographic map of the eye.

The predetermined reference pattern is preferably an artificial pattern as hereinbefore described. It may, however, be a natural pattern as hereinbefore described.

According to yet a further aspect of the invention there is provided an ophthalmic applicator that is shaped, configured and dimensioned for applying at least one predetermined reference pattern to the surface of an eye.

The applicator may include at least one impression means defining a surface having a predetermined shape and adapted to be coated with a staining substance, for impression upon an eye thereby to leave an imprint of the staining substance on the eye, having the predetermined shape. Instead, or in addition, the applicator may include mask or stencil means, defining at least one window having a predetermined shape, through which a staining substance may be applied to an eye, thereby to leave an imprint of the staining substance on the eye, having the predetermined shape. The impression surface(s) and/or window(s) may define character(s) recognizable by OCR hardware and/or software means. The applicator may include three angularly spaced limbs extending from handle means, each limb terminating in an impression means or mask means as hereinbefore described.

### BRIEF DESCRIPTION OF THE DRAWINGS

The invention will now be described further by way of non-limiting example with reference to the accompanying diagrammatic drawings. In the drawings:

FIG. 1 shows, schematically, a three-dimensional view of an embodiment of an ophthalmic applicator according to the invention.

FIG. 2 shows, schematically, a corneal bitmap image showing markings applied by the applicator of FIG. 1 to the periphery of a cornea.

FIG. 3 shows, schematically, the components of a ophthalmic surgery system in accordance with the principles of the present invention.

### BEST MODE FOR CARRYING OUT THE INVENTION

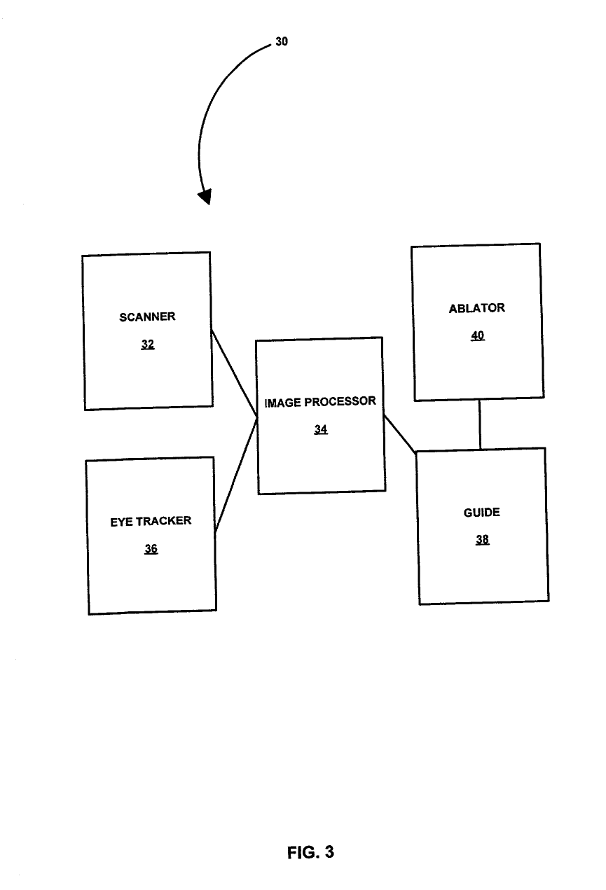

An example of an ophthalmic surgery system 30 according to the invention is first described, as shown in FIG. 3.

The system includes, firstly, scanning means configured to derive a topographic map of a patient's eye, and an image of the eye. The scanning means, such as scanner 32, may be a development of a conventional topographer, equipped to capture a bitmap image of the eye together with a topographic map thereof.

The system further includes image processing means, such as image processor 34. In the embodiment of the system currently under consideration, the image processing means is in the form of software with which the scanning means (i.e., the developed topographer) is programmed. The software includes routines capable of identifying a predetermined reference pattern in the image of the eye that has been captured by the scanning means. In practice, the pattern will typically comprise reference markings in the form of characters, so the software may include conventional OCR software for identifying such characters.

For enhanced precision, it is envisioned that the software will be capable of identifying and locating specific portions of characters present in the image and registering the location and/or orientation of the specific portions. For example, if the characters making up the pattern are the characters “-”, “X” and “O”, the software may be designed to identify and locate one end of the “-”, and the center-points of the “X” and the “O”.

The software of the image processing means is further adapted to correlate the location and/or orientation of the identified pattern (or specific portions thereof) with corresponding coordinates of the topographic map representing the pattern's position and/or orientation on the topographic map. Consequently, the location and/or orientation of the identified pattern will also be correlated with data (e.g., elevation data) associated with the coordinates on the topographic map. The software will then compile resulting correlation data cross-referencing the location and/or orientation of the identified pattern with the corresponding coordinates and data on the topographic map.

The system further includes eye-tracking means configured to track the predetermined reference pattern, such as eye tracker 36. Preferably, the eye-tracking means will continually capture images (e.g., bitmap images) of the eye, at a frequency of 10 images per second or greater. Again, the eye-tracking means will typically be programmed with optical character recognition software, for identifying in the captured images the characters (or portions thereof) making up the predetermined reference pattern, and registering their location and/or orientation.

The embodiment of the system described here by way of example also has guidance means such as guide 38, for guiding an excimer laser system, or other ablator 40, to apply a refractive ablation to a locus of an eye. The guidance means typically includes software adapted to determine the topographic map coordinates of the locus relative to those of the characters or portions of characters making up the predetermined reference pattern. For achieving this purpose, the software accesses the correlation data produced by the image processing means.

The software of the guidance means is adapted also to access precalculated treatment data for the topographic map coordinates from a treatment data file (in the form of a laser-shot file, as will be further discussed herein). The software then has at its disposal sufficient information to guide the laser to perform the precalculated treatment at the locus.

In use of such a system, the following series of steps will typically be carried out. Firstly, and in accordance with conventional practice, a patient for refractive surgery has a topographic map of an eye to be treated compiled. A conventional topographer may be used to compile the map. This step can be carried out days or weeks prior to the surgery, for example in an ophthalmologist's consulting rooms.

The topographic map is then employed to build up a so-called “laser-shot file” (i.e., a calculated treatment for a particular eye, based on the topographic map). The topographic map and laser-shot file are then either saved to disk in the usual text file format, for transfer to the laser system, or are downloaded to the laser system via a network or the Internet.

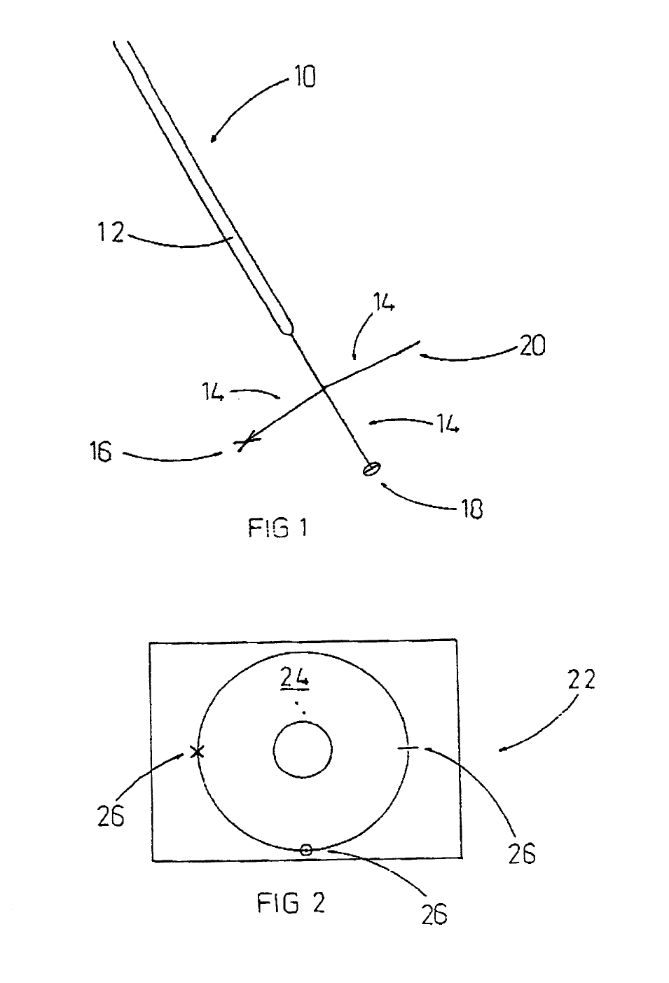

On the day of surgery, and typically immediately prior to the surgery, an ophthalmic applicator such as that shown in FIG. 1 is used to apply a predetermined reference pattern in the form of reference markings to the surface of the patient's eye. The predetermined reference pattern may be any desired number of reference markings useful for registering the location and/or orientation of the predetermined reference pattern, but it is currently preferred to use three reference markings. It is also preferred to apply the reference markings at the periphery of the patient's cornea.

One embodiment of an applicator, in accordance with the principles of the present invention, indicated generally by reference numeral 10, shown in FIG. 1, includes handle means 12, from which extend three rectangularly spaced limbs 14. Impression means 16, 18, 20 are provided at the ends of the limbs 14. The impression means 16 at the “nine o'clock” position of the applicator defines an impression surface in the form of an “X”. The impression means 18 at the “six o'clock” position defines an impression surface in the form of an “O”. The impression means 20 in the “three o'clock” position defines an impression surface in the form of a “-”. The shapes of the impression surfaces are selected to enable them to impress characters recognizable by the OCR software of the scanning means and eye-tracking means.

Before use, the impression surfaces of the applicator 10 are coated with a physiologically acceptable staining substance e.g. methylene blue. The applicator 10 is then applied to the cornea of the patient's eye, so that the impression surfaces 16, 18, 20 align approximately with the periphery of the patient's cornea and in approximate register with the three o'clock, six o'clock and nine o'clock positions of the patient's cornea. The reference markings in the form of the above three characters (“X”, “O”, and “-”) are then imprinted on the surface of the cornea in these positions. These positions are selected because they provide a convenient reference framework within which the surgery will be conducted, but it is of course equally permissible for the markings to be applied in other positions.

A further topographic scan of the patient's eye is then made, using the scanning means described above (which includes a topographer). The scanning means simultaneously captures a bitmap image of the eye.

Referring to FIG. 2 of the drawings, reference numeral 22 indicates generally a schematic diagram of a bitmap image of a cornea 24, such as that which might be captured by scanning means and/or eye-tracking means forming part of the system described above. Reference markings 26 which have been applied with an applicator such as that shown in FIG. 1 are shown on the periphery of the cornea in the three o'clock, six o'clock and nine o'clock positions.

As previously discussed, the software of the scanning means is customized to search for specific portions of the three characters (e.g., “X”, “O”, and “-”), and to register the location and/or orientation of the portions of the characters in relation to the topographic map of the cornea which is built up by the topographer.

Details of the location and/or orientation of the portions of the characters in relation to the corneal elevations for specific coordinates on the cornea, and other data contained in the topographic map, is then transferred to the laser system, where it can be read by the eyetracking means.

The original topographic map and the laser-shot file with processed elevation information are then digitally correlated with the new topographic scan, and the laser treatment is then placed in appropriate relation to the reference markings on the cornea.

While the laser treatment is carried out, the software of the eye-tracking means continually searches for the reference markings on the cornea and uses these reference markings as reference points within which and in relation to which the laser treatment is applied.

The inventor believes that the methods and systems as described and illustrated herein have advantages over conventional methods and systems for performing intended custom corneal ablations with lasers, and for eye-tracking.

For example, current conventional methods of eye-tracking, and hence laser treatment, rely on mathematical or manual fixing of approximate pupillary or corneal centers. Reliance is placed on an approximate visual axis. Conventional eye-trackers thus assume a visual axis of an eye, and a calculated treatment for a patient based on the topography of the patient's eye is then applied to the patient's eye, with reference to the approximate visual axis. However, there is no direct coordination of the topographical data, point for point, with the laser treatment. With the methods and systems as illustrated and described herein, the markings applied to the eye serve as a link to draw together the topographic map and the laser-guidance system.

Since the methods and systems as illustrated and described do not rely on the determination of a pupillary or corneal center point, the inventor believes that they will be of particular use in cases where the laser irradiation surface of a cornea has poor resolution (for example in the LASIK procedure after forming of the flap), and also in cases where the pupil is irregular, or where torsion of the eye occurs during surgery.

Applications of the eye-tracking methods and systems as described and illustrated are not limited to refractive eye surgery. For example, in other ophthalmic applications the methods and systems described permit real time eye-tracking, and can assist in photodynamic therapy and laser iridopuncture, facilitating precise placing and configuration of laser radiation in an operation zone.

Outside the field of ophthalmology, the eye-tracking methods and systems as described and illustrated may also find application in human-computer interaction, e.g. as input devices in user-computer dialogue. For example, the eye-tracking methods and systems could be employed to create interfaces between computer users and computers, to permit the eyes of such users to function as pointing, selection, dragging, etc. devices for software created objects, and serve other computer input functions. Eye-tracking to facilitate computer input in this way may be particularly beneficial for pilots or disabled people.

Application of the eye-tracking methods and systems according to the invention in such other fields falls within the scope of the invention. Moreover, those skilled in the art will appreciate that various other applications for the methods and systems disclosed herein are also within the scope of the invention.

It will be appreciated by those skilled in the art that illustrated embodiments herein described are not intended to limit the invention or the scope of the appended claims. Various combinations and modifications of the preferred embodiments could be made without departing from the scope of the present invention and all such modifications are within the scope of the present invention. For example, it is understood that while the methods and apparatus of the present invention have been described in relation to an excimer laser interacting with a guidance means directed by a topographic map correlated to a predetermined reference pattern, one of skill in the art will recognize that the present invention may be utilized with a wide variety of ablation means and/or scanning and correlation devices.

Thus, while certain representative embodiments and details have been shown for purposes of illustrating the invention, it will be apparent to those skilled in the art that various changes in the invention disclosed herein may be made without departing from the scope of the invention, which is defined in the appended claims.

What is claimed is:

1. A method of notionally superimposing a topographic map of an eye upon a cornea of an eye, comprising:
applying a predetermined reference pattern to an eye;
scanning the eye to derive a topographic map of thereof and capture data representing the location and/or orientation of said pattern upon the eye;
correlating said location and/or orientation of said pattern with corresponding coordinates of said topographic map representing said pattern's location and/or orientation on said topographic map, and/or with data associated with said coordinates on said topographic map;
tracking said pattern continually, to produce a real-time, intermittent update of said location and/or orientation of said pattern; and
notionally projecting said location and/or orientation of said topographic map upon the eye by mapping said corresponding coordinates of said topographic map to said physical location and/or orientation of said pattern.

2. The method according to claim 1, wherein applying said predetermined reference pattern comprises applying a staining substance to the eye at selected points to form a predetermined reference pattern.

3. The method according to claim 2, wherein said staining substance is applied by touching an applicator coated with said staining substance to a surface of the eye.

4. The method according to claim 2, wherein said staining substance is applied by applying a stencil to a surface of the eye and applying staining substance therethrough.

5. The method according to claim 2, wherein said staining substance comprises methylene blue.

6. The method according to claim 1, wherein applying said predetermined reference pattern comprises inserting at least one suture into a surface of the eye.

7. The method according to claim 1, wherein applying said predetermined reference pattern comprises applying at least one patch to a surface of the eye.

8. The method according to claim 1, wherein applying said predetermined reference pattern comprises applying a template to a surface of the eye.

9. The method according to claim 1, wherein applying said predetermined reference pattern comprises applying at least one uniquely identifiable reference mark.

10. The method according to claim 9, wherein correlating said location and/or orientation of said pattern with corresponding coordinates of said topographic map comprises recognizing said uniquely identifiable mark and correlating a point on said topographic map to said uniquely identifiable mark.

11. The method according to claim 10, wherein recognizing said uniquely identifiable mark is accomplished using DSP or OCR.

12. The method according to claim 1, wherein said data associated with said coordinates is selected from the group consisting of elevation data, placido data, and wavefront data.

13. The method according to claim 1, wherein tracking said pattern continually, to produce a real-time, intermittent update of said location and/or orientation of said pattern, comprises repeatedly scanning the surface of the eye and repeatedly said correlating said location and/or orientation of said pattern with corresponding coordinates of said topographic map.

14. The method according to claim 13, wherein said repeatedly scanning comprises scanning the surface of the eye at least about 10 times per second.

15. An automated ophthalmic surgery system for conducting topography-guided ablation of an eye, comprising:
a scanner configured to derive a topographic map of an eye, and capture an image of the eye;
an image processor configured to (i) identify a predetermined reference pattern in said image of the eye, (ii) correlate a location and/or orientation of said predetermined reference pattern with corresponding coordinates of said topographic map representing said predetermined reference pattern's position and/or orientation on said topographic map, and/or with data associated with coordinates on said topographic map, and (iii) compile resulting correlation data;
an eye tracker configured to track said predetermined reference pattern; and
a guide adapted to guide ophthalmic ablation means in application of surgical treatment to a locus of the eye.

16. The automated ophthalmic surgery system of claim 15, wherein said guide functions by (i) determining said topographic map coordinates of said locus relative to those of said predetermined reference pattern by means of said correlation data produced by said image processor, (ii) accessing predetermined treatment data for said topographic map coordinates from a treatment data file, and (iii) guiding said ophthalmic ablator to perform said predetermined treatment.

17. The automated ophthalmic surgery system of claim 15, wherein said data associated with said coordinates is selected from the group consisting of elevation data, placido data, and wavefront data.

18. The automated ophthalmic surgery system of claim 15, wherein said ophthalmic ablation means comprises an excimer laser.

19. The automated ophthalmic surgery system of claim 15, wherein said predetermined reference pattern comprises a staining substance applied to the eye at selected points.

20. The automated ophthalmic surgery system of claim 19, wherein said staining substance comprises methylene blue.

21. The automated ophthalmic surgery system of claim 15, wherein said predetermined reference pattern comprises at least one suture inserted into a surface of the eye or at least one patch applied to a surface of the eye.

22. The automated ophthalmic surgery system of claim 15, wherein said predetermined reference pattern comprises a template applied to a surface of the eye or a visualizable characteristic of the eye.

23. The automated ophthalmic surgery system of claim 22, wherein said visualizable reference pattern comprises a pattern of conjunctival vessels of the eye or an iris nevi pattern of the eye.

24. The automated ophthalmic surgery system of claim 15, wherein said predetermined reference pattern comprises at least one uniquely identifiable reference mark.

25. The automated ophthalmic surgery system of claim 25, wherein said image processor is configured to correlate said location and/or orientation of said pattern with corresponding coordinates of said topographic map through a process comprising recognizing said uniquely identifiable mark and correlating a point on said topographic map to said uniquely identifiable mark.

26. The automated ophthalmic surgery system of claim 25, wherein said image processor recognizes said uniquely identifiable mark using DSP or OCR.

27. The automated ophthalmic surgery system of claim 15, wherein said eye tracker functions by deriving successive images of the eye and identifying in each of said successive images said predetermined reference pattern and its location and/or orientation.

28. The automated ophthalmic surgery system of claim 27, wherein deriving successive images of the eye occurs comprises repeatedly scanning the surface of the eye.

29. The automated ophthalmic surgery system of claim 28, wherein said repeatedly scanning comprises scanning the surface of the eye at least about 10 times per second.

30. A data compilation system for compiling data useful for topography-guided ablation of an eye, comprising:
a scanner configured to derive a topographic map of an eye and to capture an image of the eye; and
an image processor configured to (i) identify a predetermined reference pattern in said image of the eye, (ii) correlate a location and/or orientation of said pattern with corresponding coordinates of said topographic map representing said pattern's position and/or orientation on said topographic map, and/or with data associated with said coordinates, and (iii) compile resulting correlation data.

31. The data compilation system of claim 30, wherein said data associated with said coordinates is selected from the group consisting of elevation data, placido data, and wavefront data.

32. The data compilation system of claim 30, wherein said predetermined reference pattern comprises a staining substance applied to the eye at selected points.

33. The data compilation system of claim 32, wherein said staining substance comprises methylene blue.

34. The data compilation system of claim 30, wherein said predetermined reference pattern comprises at least one suture inserted into a surface of the eye, at least one patch applied to a surface of the eye, or a template applied to a surface of the eye.

35. The data compilation system of claim 30, wherein said predetermined reference pattern comprises a visualizable characteristic of the eye.

36. The data compilation system of claim 35, wherein said visualizable characteristic comprises a pattern of conjunctival vessels of the eye.

37. The data compilation system of claim 35, wherein said visualizable characteristic comprises an iris nevi pattern of the eye.

38. The data compilation system of claim 30, wherein said predetermined reference pattern comprises at least one uniquely identifiable reference mark.

39. The data compilation system of claim 38, wherein said image processor is configured to correlate said location and/or orientation of said pattern with corresponding coordinates of said topographic map through a process comprising recognizing said uniquely identifiable mark and correlating a point on said topographic map to said uniquely identifiable mark.

40. The data compilation system of claim 38, wherein said image processor recognizes said uniquely identifiable mark using DSP or OCR.

41. A method of performing an automated ablation treatment of an eye in register with the eye concerned, comprising:
calculating a treatment applicable at a locus of an eye corresponding to coordinates on a topographic map, and compiling a laser-shot file correlating data relating to said treatment with said coordinates;
correlating a location and/or orientation of a predetermined reference pattern on a surface of the eye with said coordinates on said topographic map representing said predetermined reference pattern's position and/or orientation on said topographic map, and/or with data associated with said coordinates;
identifying said predetermined reference pattern on the surface of the eye and continually tracking its movement thereby to track notional movement of said topographic map and, consequently, said locus of the eye; and
performing said treatment calculated for said locus, at said locus.

42. The method according to claim 41, wherein correlating said location and/or orientation of said predetermined reference pattern on the surface of the eye with said coordinates on said topographic map comprises:
capturing an image of the eye; and
employing an image processor means to (i) identify said predetermined reference pattern in said image of the eye, (ii) correlate said location and/or orientation of said predetermined reference pattern with corresponding coordinates of said topographic map representing said predetermined reference pattern's position and/or orientation on said topographic map, and/or with data associated with said coordinates, and (iii) compile resulting correlation data.

43. The method according to claim 41, wherein said image processor identifies said predetermined reference pattern using DSP or OCR.

44. The method according to claim 41, wherein said image of the eye is selected from the group consisting of a digital image, a photograph, and a video still

45. The method according to claim 44, wherein said image of the eye is a digital image comprising a bitmap.

46. The method according to claim 49, wherein data associated with said coordinates is selected from the group consisting of elevation data, placido data, and wavefront data.

47. The method according to claim 41, wherein performing said treatment comprises performing ophthalmic ablation at said locus.

48. The method according to claim 47, wherein said ophthalmic ablation is performed with an excimer laser.

49. The method according to claim 41, further comprising:
disposing said predetermined reference pattern on the surface of the eye by applying a staining substance to the surface at at least one selected point.

50. The method according to claim 49, wherein said staining substance comprises methylene blue.

51. The method according to claim 50, wherein disposing said staining substance comprises touching an applicator coated with said staining substance to the surface or applying a stencil to a surface of the eye and applying staining substance therethrough.

52. The method according to claim 41, further comprising:
disposing said predetermined reference pattern on the surface of the eye by inserting at least one suture into the surface or by applying at least one patch to the surface.

53. The method according to claim 49, further comprising:
disposing said predetermined reference pattern on the surface of the eye by applying a template to the surface.

54. The method according to claim 41, wherein said predetermined reference pattern comprises a visualizable characteristic of the eye.

55. The method according to claim 54, wherein said visualizable characteristic comprises a pattern of conjunctival vessels of the eye or an iris nevi pattern of the eye.

56. The method according to claim 41, wherein said predetermined reference pattern comprises at least one uniquely identifiable reference mark.

57. A method of tracking an eye, comprising:
configuring an eye-tracking system to recognize at least one predetermined reference pattern;
applying said predetermined pattern directly to a surface of an eye; and
continually tracking said predetermined reference pattern with said eye-tracking system.

58. The method according to claim 57, wherein applying said predetermined reference pattern comprises applying a staining substance to the surface of the eye at at least one selected point.

59. The method according to claim 58, wherein said staining substance is applied by touching an applicator coated with said staining substance to a surface of the eye.

60. The method according to claim 58, wherein said staining substance is applied by applying a stencil to a surface of the eye and applying staining substance therethrough.

61. The method according to claim 58, wherein said staining substance comprises methylene blue.

62. The method according to claim 57, wherein applying said predetermined reference pattern comprises inserting at least one suture into a surface of the eye, applying at least one patch to a surface of the eye, or applying a template to a surface of the eye.

63. The method according to claim 57, wherein said applying predetermined reference pattern comprises applying at least one uniquely identifiable reference mark.

64. The method according to claim 63, wherein said eye-tracking system recognizes said uniquely identifiable mark using DSP or OCR.

65. The method according to claim 57, wherein continually tracking said predetermined reference pattern with said eye-tracking system comprises deriving successive images of the eye and identifying in each of said successive images said predetermined reference pattern and its location and/or orientation.

66. The method according to claim 65, wherein deriving successive images of the eye comprises repeatedly scanning the surface of the eye.

67. The method according to claim 66, wherein said repeatedly scanning comprises scanning the surface of the eye at least about 10 times per second.

68. An applicator for applying a predetermined reference pattern to the surface of an eye, comprising:
a handle;
at least one limb extending from said handle;
a first impression surface disposed on said at least one limb, said first impression surface configured to impress a reference marking onto a surface of an eye.

69. The applicator of claim 68, further comprising a staining substance disposed on said first impression surface.

70. The applicator of claim 69, wherein said staining substance is methylene blue.

71. The applicator of claim 68, further comprising:
a second limb extending from said handle;
a second impression surface disposed on said second limb, said second impression surface configured to impress a reference marking onto a surface of an eye.

72. The applicator of claim 71, further comprising:
a third limb extending from said handle;
a third impression surface disposed on said third limb, said third impression surface configured to impress a reference marking onto a surface of an eye.
